(19) United States
(12) Patent Application Publication
(10) Pub. No.: US 2007/0197843 A1
(43) Pub. Date: Aug. 23, 2007

(54) Method for purification of 1,1-dichloroethane and process for production of 1,1-difluroethane using this method

(75) Inventor: Hiromoto Ohno, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., KAWASAKI-SHI (JP)

(21) Appl. No.: 10/593,289

(22) PCT Filed: Apr. 13, 2005
(86) PCT No.: PCT/JP05/07492

§ 371 Date: Sep. 18, 2006

Related U.S. Application Data
(60) Provisional application No. 60/567,811, filed on May 5, 2004.

(30) Foreign Application Priority Data
Apr. 26, 2004 (JP) 2004-129917

(51) Int. Cl. C07C 17/38; C07C 19/08

(52) U.S. Cl. 570/163; 570/238; 977/900

(57) ABSTRACT

1,1-dichloroethane containing a compound having a nitro group and/or a hydroxyl group as a stabilizer is brought into contact with zeolite having an average pore size of 3.4 to 11 Å and/or a carbonaceous adsorbent having an average pore size of 3.4 to 11 Å in a liquid phase. A stabilizer contained in 1,1-dichloroethane is efficiently removed by a simple and convenient method and 1,1-difluoroethane can be economically produced.

### CROSS-REFERENCE TO RELATED APPLICATION

This application is an application filed under 35 U.S.C. § 111(a) claiming benefit pursuant to 35 U.S.C. § 119(e)(1) of the filing date of the Provisional Application 60/567,811 filed May 5, 2004, pursuant to 35 § 111(b).

### TECHNICAL FIELD

The present invention relates to a method for purification of 1,1-dichloroethane and a process for production of 1,1-difluoroethane using that method.

### BACKGROUND Art

As methods for the production of 1,1-dichloroethane (CH₃CHCl₂), for example:

(1) a method of reacting acetylene and hydrogen chloride to synthesize vinyl chloride (CH₂═CHCl) and further adding hydrogen chloride;

(2) a method of causing phosphorus pentachloride to act upon acetoaldehyde; and so on are known. In these methods, in order to secure the stability of the production processes and the stability of the products, a stabilizer is incorporated in an amount of several hundreds to several thousands of mass ppm in general.

On the other hand, 1,1-difluoroethane (CH₃CHF₂) produced using for example 1,1-dichloroethane as a raw material is attracting attention as for example a low temperature use refrigerant or etching gas.

As production methods and purification methods of 1,1-difluoroethane, the following methods have heretofore been known: For example:

(3) a production method reducing a chlorofluorohydrocarbon by hydrogen in the presence of a catalyst (see Japanese Unexamined Patent Publication No. 7-126197); and

(4) a production method reacting 1,1-dichloroethane and hydrogen fluoride in the presence of a catalyst (see Japanese Unexamined Patent Publication No. 6-228021); and so on.

For example, the 1,1-difluoroethane produced by using the methods of (3) and (4) described above contains various impurities including saturated compounds and unsaturated compounds etc. generated during the production, for example, hydrocarbons (HC), hydrochlorocarbons (HCC), chlorofluorocarbons (CFC), hydrochlorofluorocarbons (HCFC), and hydrofluorocarbons (HFC).

Especially, in the method of reacting 1,1-dichloroethane and hydrogen fluoride in the presence of a fluorination catalyst, one of the reaction materials, that is, the 1,1-dichloroethane, contains a stabilizer in an amount of about several hundreds to several thousands mass ppm incorporated in order to suppress the generation of an acidic component due to decomposition. As the stabilizer, there can be mentioned for example nitromethane, nitroethane, nitrocresol, nitrotoluene, nitrophenol, phenol, cresol, 2,6-di-butyl-p-cresol, and aminomethylphenol as compounds having nitro groups and/or hydroxyl groups. When these stabilizers are not contained, the 1,1-dichloroethane lacks stability, and side effects such as generation of the acidic component due to the decomposition proceeds.

However, the stabilizer contained in 1,1-dichloroethane becomes a cause of deterioration of the activity of the catalyst used when producing the 1,1-difluoroethane, the catalyst life becomes short, and other problems arise. Therefore, desirably such a stabilizer is not contained.

Accordingly, for example, the stabilizer may be removed before reaction, but a conventional removal method by conventional fractional distillation has the problem that the operation is troublesome, so the cost is very expensive.

### DISCLOSURE OF INVENTION

The present invention was made under such a background and has as the problem thereof to provide a method for the purification of 1,1-dichloroethane able to remove the stabilizer contained in the 1,1-dichloroethane, easy to operation, and industrially possible and a process for the production of 1,1-difluoroethane using this purification method.

The inventors engaged in intensive studies to solve the above problem and as a result found that by bringing 1,1-dichloroethane containing a stabilizer into contact with zeolite having an average pore size of 3.4 to 11 Å and/or a carbonaceous adsorbent having an average pore size of 3.4 to 11 Å in a liquid phase, the contained stabilizer can be reduced. Further, they found that by using 1,1-dichloroethane containing a reduced amount of stabilizer as the raw material, the advantages such as the prolongation of the catalyst life are obtained and the 1,1-difluoroethane can be efficiently and economically produced and thereby completed the present invention.

Accordingly, the present invention provides a method for the purification of 1,1-dichloroethane and a process for the production of 1,1-difluoroethane using the purification method shown in for example the following [1] to [13].

[1] A method for the purification of 1,1-dichloroethane comprising bringing 1,1-dichloroethane containing a compound having a nitro group and/or a hydroxyl group as a stabilizer into contact with zeolite having an average pore size of 3.4 to 11 Å and/or a carbonaceous adsorbent having an average pore size of 3.4 to 11 Å in a liquid phase to reduce the stabilizer.

[2] A method for the purification of 1,1-dichloroethane as described in the above [1], wherein an Si/Al ratio of the zeolite is 2 or less.

[3] A method for the purification of 1,1-dichloroethane described in the above [1] or [2], wherein the zeolite is at least one type selected from a group consisting of Molecular Sieve 4A, Molecular Sieve 5A, Molecular Sieve 10X, and Molecular Sieve 13X.

[4] A method for the purification of 1,1-dichloroethane described in the above [1], wherein the carbonaceous adsorbent is Molecular Sieving Carbon 4A and/or Molecular Sieving Carbon 5A.

[5] A method for the purification of 1,1-dichloroethane described in any one of the above [1] to [4], wherein a temperature for bringing the 1,1-dichloroethane containing the compound having the nitro group and/or the hydroxyl group as the stabilizer into contact with the zeolite and/or carbonaceous adsorbent is within a range of from −20 to +60° C.

[6] A method for the purification of 1,1-dichloroethane described in any one of the above [1] to [5], wherein a pressure for bringing the 1,1-dichloroethane containing the compound having the nitro group and/or the hydroxyl group as the stabilizer into contact with the zeolite and/or carbonaceous adsorbent is within a range of from 0 to 1 MPa.

[7] A method for the production of 1,1-difluoroethane comprising using as a reaction raw material 1,1-dichloroethane reduced in amount of a compound having a nitro group and/or a hydroxyl group obtained by using the purification method described in any one of the above [1] to [6] contained as a stabilizer.

[8] A process for the production of 1,1-difluoroethane comprising the following three steps:

(1) a step of using the purification method described in any one of the above [1] to [6] to reduce a compound having a nitro group and/or a hydroxyl group contained as a stabilizer in 1,1-dichloroethane;

(2) a step of reacting the 1,1-dichloroethane reduced in amount of the compound having the nitro group and/or the hydroxyl group after the step of (1) with hydrogen fluoride in a gaseous phase in the presence of a fluorination catalyst to obtain a gas mixture mainly containing 1,1-difluoroethane; and

(3) a step of separating the gas mixture mainly containing the 1,1-difluoroethane obtained in the step of (2) and recirculating at least part of an unreacted product to the step (2).

[9] A process for the production of 1,1-difluoroethane described in the above [8], wherein the step (2) is conducted by using 1,1-dichloroethane reduced in total content of the compound having the nitro group and/or the hydroxyl group obtained by the step of the above (1) to 30 mass ppm or less.

[10] A process for the production of 1,1-difluoroethane described in the above [8], wherein the step (2) is conducted by using 1,1-dichloroethane reduced in total content of the compound having the nitro group and/or the hydroxyl group obtained by the step of the above (1) to 10 mass ppm or less.

[11] A process for the production of 1,1-difluoroethane described in any one of [7] to [10], wherein the compound having the nitro group and/or the hydroxyl group is at least one type of compound selected from a group consisting of nitro methane, nitro ethane, nitro cresol, nitro toluene, nitro phenol, phenol, cresol, 2, 6-di-butyl-p-cresol, and aminomethylphenol.

[12] A process for the production of 1,1-difluoroethane described in the above [8], wherein the fluorination catalyst used in the step of the above (2) contains at least one type of element selected from a group consisting of Cu, Mg, Zn, Pb, Cr, Al, In, Bi, Co, and Ni, and the contact temperature is 100 to 350° C.

[13] A process for the production of 1,1-difluoroethane described in the above [8], wherein the unreacted product recirculated to the step (2) in the step of the above (3) is at least one type of compound selected from a group consisting of 1-chloro-1-fluoroethane, 1,1-dichloroethane, and hydrogen fluoride.

According to the present invention, the stabilizer is efficiently removed from 1,1-dichloroethane containing the stabilizer by a simple and convenient method, the deterioration etc. of the catalyst can be prevented, 1,1-difluoroethane can be economically produced, and the obtained 1,1-difluoroethane can be used as a low temperature use refrigerant or etching gas.

Best Mode for Carrying Out the Invention

Preferred embodiments of the present invention will be explained in detail below.

As mentioned above, as the method for the production of 1,1-dichloroethane, for example (1) the method of synthesizing vinyl chloride by a reaction of acetylene and hydrogen chloride and further adding hydrogen chloride thereto, (2) the method of causing phosphorus pentachloride to act upon acetoaldehyde, etc. are known, but for the above-mentioned reasons, a stabilizer is contained in an amount of about several hundreds to several thousands of mass ppm. The stabilizer becomes a cause of deterioration of the catalytic activity even if minute in amount, so the stabilizer is desirably reduced before the reaction as much as possible.

The inventors engaged in extensive studies to develop a method of reducing a stabilizer contained in 1,1-dichloroethane that is easy in operation, economical, and industrially workable and as a result discovered that the stabilizer can be reduced by bringing the 1,1-dichloroethane containing the stabilizer into contact with zeolite having an average pore size of 3.4 to 11 Å and/or a carbonaceous adsorbent having an average pore size of 3.4 to 11 Å in a liquid phase.

The zeolite used in the purification method of 1,1-dichloroethane of the present invention should be one having an average pore size of 3.4 to 11 Å, preferably 3.4 to 10 Å. Zeolite having an average pore size larger than 11 Å has an increased adsorption of 1,1-dichloroethane, while zeolite having an average pore size smaller than 3.4 Å becomes poor in ability of adsorbing the stabilizer.

Further, the Si (silica)/Al (aluminum) ratio of the zeolite is preferably 2 or less. When the Si/Al ratio is larger than 2, the stabilizer is not selectively adsorbed in some cases. As the zeolite, preferably use is made of at least one type of zeolite selected from a group consisting of Molecular Sieve 4A (MS-4A), Molecular Sieve 5A (MS-5A), Molecular Sieve 10X (MS-10X), and Molecular Sieve 13X (MS-13X). By using these zeolites, the moisture of the 1,1-dichloroethane can be simultaneously reduced. If 1,1-dichloroethane containing moisture is heated as it is, acetoaldehyde is produced, so this is not preferred.

The carbonaceous adsorbent preferably has an average pore size of 3.4 to 11 Å. A carbonaceous adsorbent having an average pore size larger than 11 Å has an increased adsorption of 1,1-dichloroethane, while a carbonaceous adsorbent having an average pore size smaller than 3.4 Å becomes poor in ability of adsorbing the stabilizer. As a carbonaceous adsorbent, preferably use is made of the Molecular Sieving Carbon 4A and/or Molecular Sieving Carbon 5A.

The zeolite and the carbonaceous adsorbent are preferably used alone if taking into account the regeneration of the adsorbent, but may be mixed as well. The ratio of mixing the zeolite and the carbonaceous adsorbent is not particularly limited, but when also taking into account the reduction of the moisture in the 1,1-dichloroethane, a mixing ratio rich in zeolite is preferred.

As the method of bringing the 1,1-dichloroethane containing the stabilizer into contact with the zeolite and/or carbonaceous adsorbent in a liquid phase, a known method such as a batch system or continuous system may be used. Industrially, a method of continuously recirculating the adsorbent on a fixed bed is preferred. The liquid standard space velocity (LHSV) can be appropriately selected according to the concentration of the stabilizer and the amount of treatment of the 1,1-dichloroethane, but usually is in the preferred range of from 1 to 80 Hr⁻¹. Further, in order to industrially conduct the method of reducing the stabilizer in the 1,1-dichloroethane, a method of providing two adsorption towers and alternately switching them to continuously perform the purification may be used as well.

The treatment temperature when purifying the 1,1-dichloroethane in the liquid phase is preferably within the range of from −20 to +60° C., more preferably within a range of from 0 to 50° C. If the treatment temperature is higher than 60° C., the capital cost will increase in the points of the heating of the apparatus and the pressure tolerance, while if the temperature is lower than −20° C., the problem arises that a cooling facility etc. become necessary. Further, the pressure is preferably within the range of from 0 to 1 MPa, more preferably within a range of from 0 to 0.6 MPa. A pressure larger than 1 MPa becomes economically disadvantageous in point of the pressure tolerance etc. of the equipment.

As explained above, by using the purification method of the present invention, the stabilizer contained in the 1,1-dichloroethane can be reduced. The purification method of the present invention is used particularly preferably for a stabilizer composed of a compound having a nitro group and/or a hydroxyl group. As the stabilizer, there can be mentioned compounds such as nitro methane, nitro ethane, nitro cresol, nitro toluene, nitro phenol, phenol, cresol, 2,6-di-butyl-p-cresol, and aminomethylphenol.

According to the method of the present invention, by bringing the 1,1-dichloroethane containing the stabilizer into contact with zeolite and/or a carbonaceous adsorbent in the liquid phase under the above described conditions, 1,1-dichloroethane reduced in total content of the stabilizer to for example 30 mass ppm or less can be obtained. Further, it is also possible to obtain 1,1-dichloroethane reduced in total content of the stabilizer to 10 mass ppm or less, more preferably 1,1-dichloroethane reduced in total content of the stabilizer to 5 mass ppm or less.

Next, an explanation will be given of the process for the production of 1,1-difluoroethane of the present invention.

The process for the production of 1,1-difluoroethane of the present invention includes the following three steps. Namely,

(1) a step of using the purification method described above to reduce a compound having a nitro group and/or a hydroxyl group contained as a stabilizer in 1,1-dichloroethane;

(2) a step of reacting the 1,1-dichloroethane reduced in amount of the compound having the nitro group and/or the hydroxyl group after the step of (1) with hydrogen fluoride in a gaseous phase in the presence of a fluorination catalyst to obtain a gas mixture mainly containing 1,1-difluoroethane; and

(3) a step of separating the gas mixture mainly containing the 1,1-difluoroethane obtained in the step of (2) and recirculating at least part of an unreacted product to the step (2).

The 1,1-dichloroethane obtained after the step of (1) is preferably 1,1-dichloroethane reduced in the total content of the compound having the nitro group and/or hydroxyl group to 30 mass ppm or less, more preferably reduced in total content of the compound having the nitro group and/or hydroxyl group to 10 mass ppm or less, particularly preferably reduced in total content of the compound having the nitro group and/or hydroxyl group to 5 mass ppm or less. If 1,1-difluoroethane is produced by using as a raw material 1,1-dichloroethane reduced in total content of the compound having the nitro group and/or hydroxyl group to 30 mass ppm or less, an increase of the service life of the catalyst used in the production process is achieved and a mixture mainly containing 1,1-difluoroethane can be efficiently and economically produced.

As the fluorination catalyst used in the production process, preferably use is made of a carrier type catalyst or a bulk type catalyst containing at least one type of element selected from a group consisting of Cu, Mg, Zn, Pb, Cr, Al, In, Bi, Co, and Ni. More preferably, it is a carrier type catalyst or a bulk type catalyst containing as a principal ingredient a trivalent chromium oxide. The reaction temperature is preferably 100 to 350° C., more preferably 150 to 300° C. The reaction pressure is preferably 0 to 1 MPa. A molar ratio between the hydrogen fluoride and the 1,1-dichloroethane (HF/CH₃CHCl₂) is preferably 3 to 20.

The mixture mainly containing the 1,1-difluoroethane obtained in the above reaction step is introduced into a distillation and purification step. In the distillation and purification step, the mixture is separated to a low boiling point fraction mainly comprised of hydrogen chloride and 1,1-difluoroethane and a high boiling point fraction mainly comprised of the unreacted product or intermediate hydrogen fluoride, 1,1-dichloroethane, and 1-chloro-1-fluoroethane. Then, the low boiling point fraction is separated into the hydrogen chloride and the target 1,1-difluoroethane. On the other hand, at least part of the hydrogen fluoride, 1,1-dichloroethane, and 1-chloro-1-fluoroethane of the high boiling point fraction is recirculated to the reaction step and reused.

The present invention will be further explained below by examples and comparative examples, but the present invention is not limited by these examples.

### RAW MATERIAL EXAMPLE 1

A commercially available 1,1-dichloroethane was analyzed by gas chromatography (column: capillary type/FID), whereupon it was found that 1091 mass ppm of nitro methane (CH₃NO₂) was contained as the stabilizer. The moisture was analyzed by a moisture meter (Karl Fischer type), whereupon it was found that 46 mass ppm of moisture was contained. The purity of the 1,1-dichloroethane was 99.76 mass%.

### RAW MATERIAL EXAMPLE 2

A commercially available 1,1-dichloroethane was analyzed in the same way as Raw Material Example 1, whereupon it was found that phenol was contained as the stabilizer, the content thereof was 542 mass ppm, 56 mass ppm of moisture was contained, and the purity of the 1,1-dichloroethane was 99.84 mass %.

### EXAMPLE 1

Into a 200 ml stainless steel cylinder, 30 g of zeolite [Molecular Sieve 5A (produced by Union Showa K.K.: average pore size: 4.2 Å, Si/Al ratio=1)] was filled. The zeolite was vacuum dried, then 100 g of the 1,1-dichloroethane shown in Raw Material Example 1 was filled while cooling the cylinder. The contents were occasionally stirred while keeping the temperature at 18° C. (room temperature). After about 6 hours, part of the liquid phase portion was sampled and analyzed by gas chromatography and a moisture meter.

As a result, the nitro methane used as the stabilizer in the 1,1-dichloroethane was reduced to 3 mass ppm, and the contained moisture was 2 mass ppm or less.

### EXAMPLE 2

The same treatment and analysis were performed by the same procedure and under the same conditions as those of Example 1 except for filling 20 g of zeolite [Molecular Sieve 13X (produced by Union Showa K.K.: average pore size of 10 Å; Si/Al ratio=0.81)]. As a result, the nitro methane used as the stabilizer contained in the 1,1-dichloroethane was found to be 1 mass ppm or less.

### EXAMPLE 3

Into a 200 ml stainless steel cylinder, 20 g of a carbonaceous adsorbent [Molecular Sieving Carbon 5A (produced by Takeda Chemical Industries, Ltd.: average pore size of 5 Å)] was filled and vacuum dried, then 90 g of the 1,1-dichloroethane shown in Raw Material Example 1 was filled while cooling the cylinder. The contents were occasionally stirred while keeping the temperature at 20° C. (room temperature). After about 4 hours, part of the liquid phase portion was sampled and analyzed under the above gas chromatography conditions. As a result, it was found that the nitro methane used as the stabilizer in the 1,1-dichloroethane was reduced to 13 mass ppm.

### COMPARATIVE EXAMPLE 1

The same treatment and analysis were performed by the same procedure and under the same conditions as those of Example 1 except for filling 30 g of zeolite [Molecular Sieve XH-9 (produced by Union Showa K.K.: average pore size of 3.2 Å; Si/Al ratio=1)]. As a result, the nitro methane used as the stabilizer contained in the 1,1-dichloroethane was found to be 1068 mass ppm, i.e., almost no reduction effect was confirmed.

### COMPARATIVE EXAMPLE 2

The same treatment and analysis were performed by the same procedure and under the same conditions as those of Example 3 except for filling 20 g of carbonaceous adsorbent [activated carbon, Particulate SHIROSAGE KL, produced by Takeda Chemical Industries, Ltd.: average pore size: 35 Å)]. As a result, almost no nitro methane used as the stabilizer in the 1,1-dichloroethane was found to be adsorbed, and a new decomposition product such as acetoaldehyde due to the side reaction by the heat of adsorption was detected.

### EXAMPLE 4

Into a 200 ml stainless steel cylinder, as the adsorbent, 15 g of the Molecular Sieve 13X described in Example 2 and 10 g of the Molecular Sieving Carbon 5A described in Example 3 were mixed and filled. The adsorbent in the cylinder was vacuum dried, then 100 g of the 1,1-dichloroethane shown in Raw Material Example 1 was filled while cooling the cylinder. The contents were occasionally stirred while keeping the temperature at 20° C. After about 6 hours, part of the liquid phase was sampled and analyzed under the above described gas chromatography conditions. As a result, the nitro methane used as the stabilizer in the 1,1-dichloroethane was found to be 1 mass ppm or less.

### EXAMPLE 5

The same treatment and analysis were performed by the same procedure and under the same conditions as those of Example 1 using the adsorbent shown in Example 1 except for using 1,1-dichloroethane containing phenol as the stabilizer shown in Raw Material Example 2. As a result, the phenol used as the stabilizer contained in the 1,1-dichloroethane was reduced to 1 mass ppm or less, and also the moisture was 3 ppm or less.

### EXAMPLE 6

Into a 2 L stainless steel cylinder, 1.90 L of the Molecular Sieve 13X (MS-13X) was filled. The 1,1-dichloroethane shown in Raw Material Example 1 was continuously fed under the conditions of 20° C. and a pressure of 0.2 MPa in a liquid phase at a linear speed of 3 L/hr. After about 3 hours after the start of feeding, the liquid at the exit was analyzed. The amount of the nitro methane used as the stabilizer in the 1,1-dichloroethane was found to be 2 mass ppm or less. This exit liquid was collected in a different vessel.

Next, use was made of an Inconel 600 type reaction vessel having an inside diameter of 1 inch and a length of 1 m filled with 80 ml of a catalyst containing as a principal ingredient trivalent chromium oxide. The temperature in the reaction vessel was controlled to 200° C. while flowing a nitrogen gas. Next, the catalyst was treated for activation while introducing hydrogen fluoride. The hydrogen fluoride was fed at 60 NL/hr under the condition of a pressure of 0.2 MPa, then the feed of the nitrogen was suspended. Thereafter, the exit liquid after treatment of the MS-13X was fed at 8 NL/hr to start the reaction. After 10 hours after the start of the reaction, the acidic component in the exit gas was washed with an aqueous alkali solution and analyzed by gas chromatography. As a result, the conversion ratio of the 1,1-dichloroethane was 99%, and the yield of the 1,1-difluoroethane was 96.3%. Thereafter, the reaction was continued, and the analysis in the same way as above description was conducted after about 100 hours and after 200 hours. As a result, the conversion ratio of the 1,1-dichloroethane was about 99%, and the yield of the 1,1-difluoroethane was about 96.2%. As apparent from these results, almost no deterioration of the catalyst was confirmed.

### COMPARATIVE EXAMPLE 3

Use was made of an Inconel 600 type reaction vessel having an inside diameter of 1 inch and a length of 1 m filled with 80 ml of the catalyst containing as a principal ingredient trivalent chromium oxide. The temperature in the reaction vessel was controlled to 200° C. while flowing a nitrogen gas. Next, the catalyst was treated for activation while introducing hydrogen fluoride. The hydrogen fluoride was fed at 60 NL/hr under the condition of a pressure of 0.2 MPa, then the feed of the nitrogen was suspended. Thereafter, the 1,1-dichloroethane (stabilizer: containing 1091 mass ppm of nitro methane) shown in Raw Material Example 1 was fed at 8 NL/hr to start the reaction. After 3 hours after the start of the reaction, the acidic component in the exit gas was washed with an aqueous alkali solution and analyzed by gas chromatography. As a result, the conversion ratio of the 1,1-dichloroethane was 98%, and the yield of the 1,1-difluoroethane was 95%. Thereafter, the reaction was continued, and the analysis in the same way as above description was conducted after about 100 hours. As a result, the conversion ratio of the 1,1-dichloroethane was 65%, and the yield of the 1,1-difluoroethane was 62%. As apparent from these results, the deterioration of the catalyst is remarkably advanced due to the stabilizer in the 1,1-dichloroethane.

### INDUSTRIAL APPLICABILITY

According to the present invention, 1,1-difluoroethane useable as a low temperature use refrigerant or etching gas can be produced industrially advantageously.

1. A method for the purification of 1,1-dichloroethane comprising bringing 1,1-dichloroethane containing a compound having a nitro group and/or a hydroxyl group as a stabilizer into contact with zeolite having an average pore size of 3.4 to 11 Å and/or a carbonaceous adsorbent having an average pore size of 3.4 to 11 Å in a liquid phase to reduce the stabilizer.

2. A method for the purification of 1,1-dichloroethane as described in claim 1, wherein an Si/Al ratio of the zeolite is 2 or less.

3. A method for the purification of 1,1-dichloroethane described in claim 1 , wherein the zeolite is at least one type selected from a group consisting of Molecular Sieve 4A, Molecular Sieve 5A, Molecular Sieve 10X, and Molecular Sieve 13X.

4. A method for the purification of 1,1-dichloroethane described in claim 1, wherein the carbonaceous adsorbent is Molecular Sieving Carbon 4A and/or Molecular Sieving Carbon 5A.

5. A method for the purification of 1,1-dichloroethane described in wherein a temperature for bringing the 1,1-dichloroethane containing the compound having the nitro group and/or the hydroxyl group as the stabilizer into contact with the zeolite and/or carbonaceous adsorbent is within a range of from −20 to +60° C.

6. A method for the purification of 1,1-dichloroethane described in claim 1, wherein a pressure for bringing the 1,1-dichloroethane containing the compound having the nitro group and/or the hydroxyl group as the stabilizer into contact with the zeolite and/or carbonaceous adsorbent is within a range of from 0 to 1 MPa.

7. A method for the production of 1,1-difluoroethane comprising using as a reaction raw material 1,1-dichloroethane reduced in amount of a compound having a nitro group and/or a hydroxyl group obtained by using the purification method described in claim 1 contained as a stabilizer.

8. A process for the production of 1,1-difluoroethane comprising the following three steps:
(1) a step of using the purification method described in claim 1 to reduce a compound having a nitro group and/or a hydroxyl group contained as a stabilizer in 1,1-dichloroethane;
(2) a step of reacting the 1,1-dichloroethane reduced in amount of the compound having the nitro group and/or the hydroxyl group after the step of (1) with hydrogen fluoride in a gaseous phase in the presence of a fluorination catalyst to obtain a gas mixture mainly containing 1,1-difluoroethane; and
(3) a step of separating the gas mixture mainly containing the 1,1-difluoroethane obtained in the step of (2) and recirculating at least part of an unreacted product to the step (2).

9. A process for the production of 1,1-difluoroethane described in claim 8, wherein the step (2) is conducted by using 1,1-dichloroethane reduced in total content of the compound having the nitro group and/or the hydroxyl group obtained by the step of the above (1) to 30 mass ppm or less.

10. A process for the production of 1,1-difluoroethane described in claim 8, wherein the step (2) is conducted by using 1,1-dichloroethane reduced in total content of the compound having the nitro group and/or the hydroxyl group obtained by the step of the above (1) to 10 mass ppm or less.

11. A process for the production of 1,1-difluoroethane described in claim 7, wherein the compound having the nitro group and/or the hydroxyl group is at least one type of compound selected from a group consisting of nitro methane, nitro ethane, nitro cresol, nitro toluene, nitro phenol, phenol, cresol, 2,6-di-butyl-p-cresol, and aminomethylphenol.

12. A process for the production of 1,1-difluoroethane described in claim 8, wherein the fluorination catalyst used in the step of the above (2) contains at least one type of element selected from a group consisting of Cu, Mg, Zn, Pb, Cr, Al, In, Bi, Co, and Ni, and the contact temperature is 100 to 350° C.

13. A process for the production of 1,1-difluoroethane described in claim 8, wherein the unreacted product recirculated to the step (2) in the step of the above (3) is at least one type of compound selected from a group consisting of 1-chloro-1-fluoroethane, 1,1-dichloroethane, and hydrogen fluoride.
